(19) United States
(12) Patent Application Publication
(10) Pub. No.: US 2022/0152090 A1
(43) Pub. Date: May 19, 2022

(54) METHOD FOR TREATING AMYLOIDOGENIC DISEASE

(71) Applicant: NATIONAL HEALTH RESEARCH INSTITUTES, Miaoli County (TW)

(72) Inventors: Shu-Yi LIN, Miaoli County (TW); Jyh-Lyh JUANG, Miaoli County (TW); Jinq-Chyi LEE, Miaoli County (TW)

(21) Appl. No.: 17/454,748

(22) Filed: Nov. 12, 2021

Related U.S. Application Data
(60) Provisional application No. 63/113,578, filed on Nov. 13, 2020.

(51) Int. Cl. A61K 31/739; C07H 13/06; A61K 31/7024; A61P 25/28

(52) U.S. Cl. CPC A61K 31/739; A61P 25/28; A61K 31/7024; C07H 13/06

(57) ABSTRACT

The present disclosure relates to a method for treating or preventing or delaying the onset or progression of an amyloidogenic disease in a subject in need, comprising administering a pharmaceutical composition comprising a therapeutically effective amount of amphiphilic liposaccharide to the subject. The present disclosure also relates to a method for selecting an agent for treating or preventing or delaying the onset or progression of an amyloidogenic disease and a novel liposaccharide.

### CROSS REFERENCE TO RELATED APPLICATION

This patent application claims the benefit of U.S. Prov. Ser. No. 63/113,578 filed 13 Nov. 2020. The application is incorporated by reference herein.

### FIELD OF THE DISCLOSURE

The present disclosure relates to a method for treating an amyloidogenic disease, particularly to a method for the clearance of amyloid-beta 42 (Aβ42).

### BACKGROUND OF THE DISCLOSURE

Non-equilibrium states, or more specifically, far-from-equilibrium conditions, have been considered critical for the modulation of biomolecular assembly in living systems. One interesting example is the role of microtubules in the formation of cytoskeleton networks, an assembly process that exhibits a non-equilibrium behavior for its self-assembly and infrequent decay. Another example is the amyloid peptides (Aβ), which undergo self-aggregation to form amyloid plaques in triggering the neurodegenerative cascade of Alzheimer's disease (AD) (E. N. Cline, M A. Bicca, K. L. Viola, W L. Klein, J. Alzheimers Dis. 2018, 64, S567-S610; A. K. Buell, Biochem. J. 2019, 476, 2677-2703; 1 Vaquer-Alicea, M. I. Diamond, Annu. Rev. Biochem. 2019, 88, 785-810). Among the various Aβ isoforms, Aβ40 and Aβ42 are the two most abundant species. Although Aβ40 is more abundant than Aβ42 in cerebrospinal fluid, Aβ42 has a higher self-aggregation potential than Aβ40 in contributing to the amyloid deposits in AD brains. From the perspective of chemical kinetics, the self-propagation of Aβ42 represents a dynamic self-assembly process involving a transition state from the pre-organized protofibrils to a stable form of fibrils (M. Ahmed, J. Davis, D. Aucoin, T Sato, S. Ahuja, S. Aimoto, J. I. Elliott, W E. Van Nostrand, S. O. Smith, Nat. Struct. Mol. Biol. 2010, 17, 561-U556; S. M Butterfield, H. A. Lashuel, Angew. Chem. Int. Ed. 2010, 49, 5628-5654; I. W Hamley, Chem. Rev. 2012, 112, 5147-5192; Z. Fu, D. Aucoin, J. Davis, W E. Van Nostrand, S. Smith, Biochemistry 2015, 54, 4197-4207; J. A. Luiken, P G. Bolhuis, J. Phys. Chem. B 2015, 119, 12568-12579; B. Morel, M P Carrasco, S. Jurado, C. Marco, F Conejero-Lara, Phys. Chem. Chem. Phys. 2018, 20, 20597-20614).

Altered homeostasis between Aβ peptide production and clearance is defined as the pathological basis for the accumulated Aβ fibrils in AD brains. However, efforts aimed at blocking Aβ42 production have not been successful, as evidenced by the fact that more than 200 clinical trials of drugs designed to decrease Aβ42 production have been terminated. There is thus a need in the art for treatment of amyloidogenic diseases.

### SUMMARY OF THE DISCLOSURE

The present disclosure provides a method for treating or preventing or delaying the onset or progression of an amyloidogenic disease in a subject in need, comprising administering a pharmaceutical composition comprising a therapeutically effective amount of amphiphilic liposaccharide to the subject.

In some embodiments of the disclosure, the amphiphilic liposaccharide is as the sole active pharmaceutical agent or agents present in a therapeutically effective amount in the pharmaceutical composition.

In some embodiments of the disclosure, the amphiphilic liposaccharide comprises a lipid A and oligosaccharide. Examples of the amphiphilic liposaccharide include but are not limited to lipopolysaccharide (LPS), monophosphoryl lipid A (MPL), 2-deoxy-6-O-(2-deoxy-3-O-[(3R)-3-hydroxytetradecanoyl]-2-{[(3R)-3-hydroxytetradecanoyl]amino}-4-O-phosphono-β-D-glucopyranosyl)-3-O-[(3R)-3-hydroxytetradecanoyl]-2-{[(3R)-3-hydroxytetradecanoyl]amino}-1-O-phosphono-α-D-glucopyranose (PIX), or 2-deoxy-6-O-(2-deoxy-3-O-[(3R)-3-hydroxytetradecanoyl]-2-{[(3R)-3-hydroxytetradecanoyl]amino}-4-O-phosphono-β-D-glucopyranosyl)-3-O-[(3R)-3-hydroxytetradecanoyl]-2-{[(3R)-3-hydroxytetradecanoyl]amino}-D-glucopyranose (PXI) or salts thereof.

In some embodiments of the disclosure, the method is for the clearance of amyloid-beta 42.

In some embodiments of the disclosure, the method is for triggering non-equilibrium co-assembly of amyloid-beta 42.

In some embodiments of the disclosure, the method is for retaining neuronal cell viability.

In some embodiments of the disclosure, the method is for rescuing Aβ42-induced apoptosis.

In some embodiments of the disclosure, the method is for enhancing endo-lysosomal clearance of amyloid-beta 42.

Examples of the amyloidogenic disease include but are not limited to Alzheimer's disease, mild cognitive impairment, Parkinson's disease with dementia, Down's syndrome, diffuse Lewy body (DLB) disease, cerebral amyloid angiopathy (CAA), vascular dementia, and mixed dementia.

The present disclosure also provides a method for selecting an agent for treating or preventing or delaying the onset or progression of an amyloidogenic disease, comprising contacting the agent with a neuronal cell, wherein if the agent enhances clearance of amyloid-beta 42 or triggers non-equilibrium co-assembly of amyloid-beta 42, the agent is a candidate agent for treating or preventing or delaying the onset or progression of an amyloidogenic disease.

In some embodiments, the agent retains neuronal cell viability, rescues Aβ42-induced apoptosis, or enhances endo-lysosomal clearance of amyloid-beta 42.

In some embodiment, the agent is an amphiphilic liposaccharide; particularly, comprising lipid A and an oligosaccharide, such as lipopolysaccharide, monophosphoryl lipid A, 2-deoxy-6-O-(2-deoxy-3-O-[(3R)-3-hydroxytetradecanoyl]-2-{[(3R)-3-hydroxytetra-decanoyl]amino}-4-O-phosphono-3-D-glucopyranosyl)-3-O-[(3R)-3-hydroxytetradecanoyl]-2-{[(3R)-3-hydroxytetradecanoyl]amino}-1-O-phosphono-α-D-glucopyranose (PIX), or 2-deoxy-6-0-(2-deoxy-3-O-[(3R)-3-hydroxytetradecanoyl]-2-{[(3R)-3-hydroxytetradecanoyl]amino}-4-O-phosphono-β-D-glucopyranosyl)-3-O-[(3R)-3-hydroxytetradecanoyl]-2-{[(3R)-3-hydroxytetradecanoyl]amino}-D-glucopyranose (PXI) or salts thereof.

The present disclosure also provides a liposaccharide of 2-deoxy-6-O-(2-deoxy-3-O-[(3R)-3-hydroxytetradecanoyl]-2-{[(3R)-3-hydroxytetradecanoyl]amino}-4-O-phosphono-β-D-glucopyranosyl)-3-O-[(3R)-3-hydroxytetradecanoyl]-2-{[(3R)-3-hydroxytetradecanoyl]amino}-1-O-phosphono-α-D-glucopyranose, or 2-deoxy-6-O-(2-deoxy-3-O-[(3R)-3-hydroxytetradecanoyl]-2-{[(3R)-3-hydroxytetradecanoyl]amino}-4-O-phosphono-β-D-glucopyranosyl)-3-O-[(3R)-3-hydroxytetradecanoyl]-2-{[(3R)-3-hydroxytetradecanoyl]amino}-D-glucopyranose, or salts thereof.

### BRIEF DESCRIPTION OF THE DRAWINGS

FIG. 1 shows a hypothesized model in which LPS might act as supramolecular bait to trigger non-equilibrium co-assembly with Aβ42 protofibrils for pro-survival effect of neuronal cells.

FIGS. 2a and 2b show confirmation of non-equilibrium interaction between LPS and Aβ42 protofibrils. (a) Oscillation of the hydrophobicity of Aβ42 was noted by repeatedly adding freshly prepared LPS or aged LPS. The y-axis of the graph represents the relative fluorescence intensity of the bis-ANS emission wavelength at 525 nm. (b) A simple illustration showing the hydrophobicity of Aβ42 protofibrils could increase and decrease by LPS influx and efflux, respectively.

FIG. 3 shows representative TEM images of insoluble shorter fibers of Aβ42 were noted after co-incubation with LPS.

FIG. 4 shows that the transient LPS-Aβ42 binding ameliorates the Aβ42-induced neuronal toxicity. The cytotoxic effect of Aβ42 on SH-SY5Y cells was found to be greatly decreased by the co-treatment of Aβ42 with LPS (comparing columns 2 and 3), but that effect was found to be completely abolished by removing the unbound LPS from the solution (comparing columns 3 and 4), whereas adding the LPS back into the solution restored the lost effect (comparing columns 4 and 5). Ctrl represents SH-SY5Y cells only.

FIGS. 5a to 5d show that Aβ42 peptides and neuronal toxicity are diminished in neuronal cells co-treated with LPS. (a) The reduced cell viability (WST assay) effect of Aβ42 on SH-SY5Y cells is significantly improved by co-treatment with LPS in a dose-dependent manner. (b) Western blotting indicating Aβ42 degradation in SH-SY5Y cells co-treated with or without LPS. (c) The LPS-induced degradation of Aβ42 peptides is suppressed by three endocytosis blockers: chlorpromazine (CPZ), methyl-β-cyclodextrin (MPCD), and dynasore. (d) The rescue effect of LPS against Aβ42 neuronal toxicity is blocked by endocytosis inhibitors. Culture medium containing 2.5% dimethyl sulfoxide (DMSO) was used for each group. ***, P<0.001.

FIGS. 6a to 6b show that matrix metalloproteinases do not contribute to the LPS-induced A3 clearance. (a) The rescue effect of LPS against Aβ42 neuronal toxicity can be observed through the treatment of the neuronal cells with pan-MMP inhibitors. (b) Western blotting of Aβ42 degradation in SH-SY5Y cells in the presence of LPS co-treated with or without pan-MMP inhibitor.

FIGS. 7a to 7b show that LPS enhances autophagy-lysosome pathway activity in neuronal cells. The co-incubation of LPS-binding Aβ42 complex and either 3MA or BA can cause a cascade of adverse consequences in neural cells: (a) decreasing the degradation of Aβ42 in neural cells; and (b) increasing the cell death of neural cells. Note that “Aβ42” is abbreviated as “Aβ” in (a) and (b). **, P<0.01; ***, P<0.001.

FIG. 8 shows that binding selectivity with LPS is identified from Aβ42, not from A040. Binding assays revealed that the adhesion weight was increased in a dose-dependent manner until a plateau was reached when 0.5-10 μg of Aβ42, but not Aβ40, were added to an LPS-coated plate. The binding percentage of Aβ42 to LPS was approximately 75%.

FIG. 9 shows that the rate constant of Aβ42 fibrillization in the absence or presence of LPS could be measured in a time-dependent manner through Aβ42 filtrate collection. After adding bio-red protein staining dye, the absorption value of the filtrate was then measured at 625 nm using an ELISA reader. The values were substituted into the calibration curve to calculate the concentrations of Aβ42 in each well. We also found that the rate constant of fibrillization showed a slight change from 0.137 μM⁻¹ min⁻¹ to 0.198 μM⁻¹ min⁻¹ (inset table) in a LPS-dependent manner.

The finding suggested that the self-propagating rate in the presence of LPS is slightly slower than Aβ42 alone.

FIG. 10 shows that the critical aggregation concentration (CAC) of LPS in the presence of either Aβ42 or Aβ40 was measured by SAXS. The upper figure shows the plots of scattering intensities as a function of q, defined by q=4πλ⁻¹ sin(θ) with the scattering angle 2θ and X-ray wavelength λ. The inset figures show these signals for nascent LPS aggregates at different concentrations in the presence of Aβ42 or Aβ40. The CAC values of the third row of figures include uncertainties (standard deviations).

FIGS. 11a to 11c show that the amphiphilic groove of Aβ42 protofibrils is required for the binding of LPS and the induction of neuronal clearance of Aβ42. (a) Two decay curves revealing that the complex formation of Aβ42 protofibrils and LPS was impeded in the presence of antagonistic O-antigen (red circles, as colistin blocked the hydrophilic domain of LPS) or lipid A (black squares, as SAuM blocked the hydrophobic domain of LPS). (b) LPS antagonists effectively suppressed the Aβ42 degradation induced by LPS in cells. (c) Pictorial illustration showing that LPS may induce an endo-lysosomal clearance of Aβ42 in neuronal cells via the formation of a non-equilibrium complex with the Aβ42 protofibrils.

FIG. 12 shows that the rescue effect of LPS against Aβ42 neuronal toxicity is blocked by LPS antagonists. The complex formation of Aβ42 protofibrils and LPS resulting in the rescue effect of neural cells was abolished when the O-antigen was antagonized by colistin (a blocker for the hydrophilic domain of LPS) or the lipid A (i.e., the active center of LPS) was antagonized by SAuM (a blocker for the hydrophobic domain of LPS).

FIG. 13 shows two Western blotting data in SH-SY5Y cells co-treated with either MPL or PIX or PXI, which was found to be able to cause Aβ42 degradation.

FIG. 14 shows synthetoutes for lipid A derivatives IX and XI (denoted as PIX and PXI) of this disclosure.

### DETAILED DESCRIPTION OF THE DISCLOSURE

Unless defined otherwise, technical and scientific terms used herein have the same meaning as commonly understood by one of ordinary skills in the art to which this disclosure pertains.

The articles “a” and “an” are used herein to refer to one or to more than one (i.e., to at least one) of the grammatical object of the article. By way of example, “an element” means one element or more than one element.

As used herein, the term “and/or” is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, “treating” or “treatment” of a state, disorder or condition includes: (1) preventing or delaying the appearance of clinical or sub-clinical symptoms of the state, disorder or condition developing in a mammal that may be afflicted with or predisposed to the state, disorder or condition but has not yet experienced or displayed clinical or subclinical symptoms of the state, disorder or condition; and/or (2) inhibiting the state, disorder or condition, i.e., arresting, reducing or delaying the development of the disease or a relapse thereof (in case of maintenance treatment) or at least one clinical or sub-clinical symptom thereof; and/or (3) relieving the disease, i.e., causing regression of the state, disorder or condition or at least one of its clinical or sub-clinical symptoms; and/or (4) causing a decrease in the severity of one or more symptoms of the disease.

The term “preventing” or “prevention” is recognized in the art, and when used in relation to a condition, it includes administering, prior to onset of the condition, an agent to reduce the frequency or severity of or delay the onset of symptoms of a medical condition in a subject relative to a subject which does not receive the agent.

The term “amyloidogenic disease” includes any disease associated with (or caused by) the formation or deposition of insoluble amyloid fibrils. Exemplary amyloidogenic disease include, but are not limited to Alzheimer's disease (AD), mild cognitive impairment, Parkinson's Disease with dementia, Down's Syndrome, Diffuse Lewy Body (DLB) disease, Cerebral Amyloid Angiopathy (CAA), vascular dementia and mixed dementia (vascular dementia and AD), amyloidosis associated with multiple myeloma, primary systemic amyloidosis (PSA), and secondary systemic amyloidosis with evidence of coexisting previous chronic inflammatory or infectious conditions. Different amyloidogenic diseases are defined or characterized by the nature of the polypeptide component of the fibrils deposited. For example, in subjects or patients having Alzheimer's disease, β-amyloid protein (e.g., wild-type, variant, or truncated β-amyloid protein) is the characterizing polypeptide component of the amyloid deposit. PSA involves the deposition of insoluble monoclonal immunoglobulin (Ig) light (L) chains or L-chain fragments in various tissues, including smooth and striated muscles, connective tissues, blood vessel walls, and peripheral nerves.

As used herein, “onset” means the occurrence in a subject of clinical symptoms associated or consistent with a diagnosis amyloidogenic disease.

As used herein, “delay” in the onset or progression of a phase consistent with amyloidogenic disease means an increase in time from a first time point to onset or worsening of a phase consistent with amyloidogenic disease, such as cognitive impairment of the Alzheimer type. For example, a delay in the onset of amyloidogenic disease means that the onset of amyloidogenic disease, as defined herein, in a subject at risk to develop amyloidogenic disease is delayed from happening at its natural time frame by at least six months, 1 year, 1½ years, 2, years, 2½ years, 3 years, 3½ years, 4 years, 4½ years, 5 years, 5½ years, 6 years, 6½ years, 7 years, 7½ years or 8 years or more, and preferably from 3 years to 8 years and more preferably for 5 years after a normal cognitive subject has been determined to be at high risk to develop amyloidogenic disease. By way of further example, a delay in the progression of cognitive impairment that may progress to amyloidogenic disease or a delay in the progression of dementia means that the rate of cognitive decline is slowed relative to its natural time frame. These determinations are performed by using appropriate statistical analysis.

As used herein, the terms “patient,” “subject,” “individual,” and the like are used interchangeably, and refer to any animal, including any vertebrate or mammal, and, in particular, a human, and can also refer to, e.g., as an individual or patient.

As used herein, the term “in need of treatment” refers to a judgment made by a caregiver (e.g., physician, nurse, nurse practitioner, or individual in the case of humans; veterinarian in the case of animals, including non-human mammals), and such judgment is that a subject requires or will benefit from treatment. This judgment is made based on a variety of factors that are in the realm of a care giver's expertise, but that include the knowledge that the subject is ill, or will be ill, as the result of a condition that is treatable by the compounds of the present disclosure.

The term “administering” includes routes of administration which allow the agent of the disclosure to perform their intended function.

As used in the present disclosure, the term “pharmaceutical composition” refers to a mixture containing a therapeutic agent administered to an animal, for example a human, for treating or eliminating a particular disease or pathological condition that the animal suffers. In some embodiments of the disclosure, the pharmaceutical composition optionally comprises pharmaceutically acceptable excipients.

The term “effective amount” of an agent as provided herein refers to a sufficient amount of the ingredient to provide the desired regulation of a desired function. As will be pointed out below, the exact amount required will vary from subject to subject, depending on the disease state, physical conditions, age, sex, species and weight of the subject, the specific identity and formulation of the composition, etc. Dosage regimens may be adjusted to induce the optimum therapeutic response. For example, several divided doses may be administered daily or the dose may be proportionally reduced as indicated by the exigencies of the therapeutic situation. Thus, it is not possible to specify an exact “effective amount.” However, an appropriate effective amount can be determined by one of ordinary skill in the art using only routine experimentation.

The term “pharmaceutically acceptable” as used herein refers to compounds, materials, compositions, and/or dosage forms which are, within the scope of sound medical judgment, suitable for use in contact with the tissues of a subject (either a human or non-human animal) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. Suitable carriers, excipients, etc. can be found in standard pharmaceutical texts.

As used herein, the term “amphiphilicity” refers to the property of one substance having both a hydrophobic site and a hydrophilic site. For example, when the medium is water, a substance having amphiphilicity forms micelle particles and the particles can be observed.

As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. For example, the pharmaceutical compositions can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, lotion, gel, ointment, or a controlled-release patch or spray applied to the skin; (4) intravaginally or intrarectally, for example, as a pessary, cream, suppository or foam; (5) sublingually; (6) ocularly; (7) transdermally; (8) transmucosally; or (9) nasally.

The present disclosure identifies a non-equilibrium state of interaction between supramolecular liposaccharide and amyloid. Structurally, the polymerized propagation of amyloid presents a specific groove that is recognized by the amphiphilicity of liposaccharide bait in a non-equilibrium manner. Functionally, the transient complex elicits a cellular response to clear extracellular amyloid deposits via an endolysosomal mechanism in neuronal cells. Since the impaired clearance of toxic amyloid deposits correlates with pathology, the non-equilibrium interaction between liposaccharide and amyloid represents a useful target for therapeutics.

Accordingly, the present disclosure provides a method for treating or preventing or delaying the onset or progression of an amyloidogenic disease in a subject in need, comprising administering a therapeutically effective amount of amphiphilic liposaccharide as an active ingredient or a pharmaceutical composition comprising the same to the subject. In another aspect, the present disclosure provides use of amphiphilic liposaccharide as an active ingredient or a pharmaceutical composition comprising the same in the manufacture of a medicament for treating or preventing or delaying the onset or progression of an amyloidogenic disease in a subject in need.

In another aspect, the present disclosure provides a pharmaceutical composition comprising a therapeutically effective amount of amphiphilic liposaccharide as an active ingredient for treating or preventing or delaying the onset or progression of an amyloidogenic disease in a subject in need.

In some embodiments of the disclosure, the amphiphilic liposaccharide acts as the sole active pharmaceutical agent or agents present in a therapeutically effective amount in the method or pharmaceutical composition as described herein. In other embodiments of the disclosure, the method comprises administering a combination comprising the pharmaceutical composition and a second pharmaceutical composition, and the second pharmaceutical composition comprises a second therapeutically active agent for treating the amyloidogenic disease, such as an antibody.

The liposaccharide disclosed herein refers to a compound comprising lipid, saccharide and lipid-saccharide conjugates. The liposaccharide may be derived from a natural source or artificial. An exemplary embodiment of the lipid is lipid A. Commonly, lipid A comprises two glucosamine (carbohydrate/sugar) units, in an β(1→6) linkage, with attached acyl chains (“fatty acids”), and normally containing one phosphate group on each carbohydrate. The lipid A as described herein may be modified. The term “oligosaccharide” refers to a carbohydrate structure having from 2 to about 7 saccharide units. The particular saccharide units employed are not critical and include, by way of example, all natural and synthetic derivatives of glucose, galactose, N-acetylglucosamine, N-acetylgalactosamine, fucose, sialic acid, 3-deoxy-D,L-octulosonic acid, and the like.

Particularly, examples of the amphiphilic liposaccharide include but are not limited to lipopolysaccharide (LPS), monophosphoryl lipid A (MPL), lipid A derivative IX (2-deoxy-6-O-(2-deoxy-3-O-[(3R)-3-hydroxytetradecanoyl]-2-{[(3R)-3-hydroxytetra-decanoyl]amino}-4-O-phosphono-β-D-glucopyranosyl)-3-O-[(3R)-3-hydroxytetradecanoyl]-2-{[(3R)-3-hydroxytetradecanoyl]amino}-1-O-phosphono-α-D-glucopyranose) (denoted as PIX), or lipid A derivative XI (2-deoxy-6-O-(2-deoxy-3-O-[(3R)-3-hydroxytetradecanoyl]-2-{[(3R)-3-hydroxytetradecanoyl]amino}-4-O-phosphono-3-D-glucopyranosyl)-3-O-[(3R)-3-hydroxytetradecanoyl]-2-{[(3R)-3-hydroxytetradecanoyl]amino}-D-glucopyranose) (denoted as PXI) or salts thereof.

The term “lipopolysaccharide” (LPS) refers to large molecules consisting of a lipid and a polysaccharide (glycophospholipid) joined by a covalent bond. LPS comprises three parts: 1) O antigen; 2) Core oligosaccharide, and 3) Lipid A. The O-antigen is a repetitive glycan polymer attached to the core oligosaccharide, and comprises the outermost domain of the LPS molecule. Core oligosaccharide attaches directly to lipid A and commonly contains sugars such as heptose and 3-deoxy-D-mannooctulosonic acid (also known as KDO, keto-deoxyoctulosonate). Lipid A is a phosphorylated glucosamine disaccharide linked to multiple fatty acids.

According to the disclosure, “monophosphoryl lipid A” is a detoxified endotoxin lipid A fraction, which lacks a saccharide and a phosphate group.

According to the disclosure, “lipid A derivatives IX and XI (denoted as PIX and PXI)” is a detoxified endotoxin lipid A fraction that belongs to one of MPL analogues that lacks two lipid chains.

The term “salts” includes any anionic and cationic complex, such as the complex formed between a cationic lipid disclosed herein and one or more anions. Non-limiting examples of anions include inorganic and organic anions, e.g., hydride, fluoride, chloride, bromide, iodide, oxalate (e.g., hemioxalate), phosphate, phosphonate, hydrogen phosphate, dihydrogen phosphate, oxide, carbonate, bicarbonate, nitrate, nitrite, nitride, bisulfate, sulfide, sulfite, bisulfate, sulfate, thiosulfate, hydrogen sulfate, borate, formate, acetate, benzoate, citrate, tartrate, lactate, acrylate, polyacrylate, fumarate, maleate, itaconate, glycolate, gluconate, malate, mandelate, tiglate, ascorbate, salicylate, polymethacrylate, perchlorate, chlorate, chlorite, hypochlorite, bromate, hypobromite, iodate, an alkylsulfonate, an arylsulfonate, arsenate, arsenite, chromate, dichromate, cyanide, cyanate, thiocyanate, hydroxide, peroxide, permanganate, and mixtures thereof. In particular embodiments, the salts of the cationic lipids disclosed herein are crystalline salts.

As disclosed herein, the pharmaceutical composition is for the clearance of amyloid-beta 42, for triggering non-equilibrium co-assembly of amyloid-beta 42, for retaining neuronal cell viability, for rescuing Aβ42-induced apoptosis, and/or for enhancing endo-lysosomal clearance of amyloid-beta 42.

As illustrated in the Examples, the amphiphilic groove of Aβ42 protofibrils is important for complexing with amphiphilic liposaccharide through a pattern of non-equilibrium behavior. Liposaccharide may actually act as a bait in attracting the Aβ42 intermediates that sometimes deviate from their supposed thermodynamic self-assembly process. With respect to functionality, such a transient supramolecule-supramolecule interaction can potently stimulate a strong cellular response toward autophagy-mediated protein degradation for Aβ42 peptides in neuronal cells. Moreover, since the oscillation of the non-equilibrium state appears to sustainably maintain the far-from-equilibrium behavior during the interaction between these two supramolecules (that is, liposaccharide and Aβ42), Aβ42 peptides are found to be persistently imported into the cells for degradation. Consequently, the extracellular Aβ42 protofibrils are eventually diminished over a prolonged incubation time with the cells. The structural-functional theory regarding the non-equilibrium state of supramolecule-supramolecule binding indicates a target for the treatment of the amyloidogenic disease.

The present disclosure also provides a method for selecting an agent for treating or preventing or delaying the onset or progression of an amyloidogenic disease, comprising contacting the agent with a neuronal cell, wherein if the agent enhances clearance of amyloid-beta 42 or triggers non-equilibrium co-assembly of amyloid-beta 42, the agent is a candidate agent for treating or preventing or delaying the onset or progression of an amyloidogenic disease.

The present disclosure also provides a liposaccharide of 2-deoxy-6-O-(2-deoxy-3-O-[(3R)-3-hydroxytetradecanoyl]-2-{[(3R)-3-hydroxytetradecanoyl]amino}-4-O-phosphono-β-D-glucopyranosyl)-3-O-[(3R)-3-hydroxytetradecanoyl]-2-{[(3R)-3-hydroxytetradecanoyl]amino}-1-O-phosphono-α-D-glucopyranose, or 2-deoxy-6-O-(2-deoxy-3-O-[(3R)-3-hydroxytetradecanoyl]-2-{[(3R)-3-hydroxytetradecanoyl]amino}-4-O-phosphono-β-D-glucopyranosyl)-3-O-[(3R)-3-hydroxytetradecanoyl]-2-{[(3R)-3-hydroxytetradecanoyl]amino}-D-glucopyranose, or salts thereof.

The following examples are provided to aid those skilled in the art in practicing the present disclosure.

### EXAMPLES

Methods

The general procedures used for preparing the transient LPS-Aβ42 complex and the validation of cell cytotoxicity were as follows. First, Aβ42 (100 μM) and LPS (1000 nM, derived from Escherichia coli O111:B4, Sigma-Aldrich) were added to each group medium containing 2.5% dimethyl sulfoxide (DMSO) and then sterilized by using UV light. The medium was then replaced and the remaining mixture was incubated for 72 hours at 37° C. in 5% CO2 humidified air. Cell viability was measured using the CCK8 (Sigma-Aldrich 96992) assay according to the manufacturer's protocol. A 0.22-μm filter (non-pyogenic, Millex®-GV) was used to remove unbound LPS, if needed, in the LPS-Aβ42 solution.

Materials and Methods

An LPS-coated plate for Aβ42 and Aβ40 binding assay. 100 mM Na₂CO₃, 20 mM EDTA, and 0.1 mL of 30 μg/mL LPS solution were added to a 96-well immunoassay plate (Costar 9018, Corning Corporation), which was then incubated at 37° C. for 3 hours. The coated plate was then washed with deionized water and dried for one day. PBS solution containing 1% BSA was then added to block the coated plate at 37° C. for 30 minutes. Finally, the coated plate was washed three times with PBS solution containing 0.1% BSA. Subsequently, different concentrations of Aβ42 protofibrils (with or without added SAuM or colistin) were added to the coated wells and incubated at 37° C. for 16 hours, before being washed three times with PBS. Bio-red protein staining dye was then added into the coated wells to assay the Aβ42 or Aβ40 concentrations of each well and measure the absorption value at 625 nm using an ELISA reader. The values were substituted into the calibration curve to calculate the concentrations of Aβ42 or Aβ40 in each well.

Polymerization rate of Aβ42. The methods for the liquid type were similar to those used for the LPS-coated plate, but a filter was used to remove Aβ42 fibers during the sample preparation. In brief, a mixture of Aβ42 and LPS was incubated at 37° C. in a time-dependent manner. Then, the Aβ42 fibers were removed using a 0.1-um filter of MWCO (Millipore MILLEX-HIP) to avoid interference. The Aβ42 filtrate was collected and dried before adding bio-red protein staining dye, and the absorption value of the filtrate was then measured at 625 nm using an ELISA reader. The values were substituted into the calibration curve to calculate the concentrations of Aβ42 in each well.

Identification of the non-equilibrium steady state between LPS and Aβ42. A RPMI medium containing Aβ42 (100 μM) and LPS (1.0 nM) was incubated at 37° C. At different time points, a small proportion of the solution was taken out to stain with a fluorescent dye, bis-ANS (1.0 μM, 4,4′-Dianilino-1,1′-binaphthyl-5,5′-disulfonic acid dipotassium salt, Sigma-Aldrich), and was immediately measured using a fluorescence spectrophotometer (Varian, Cary Eclipse, excitation wavelength at 390 nm).

Transmission electron microscopy (TEM). The mixtures of LPS and individual Aβ42 were prepared in deionized water. Samples were mounted on a 400-mesh Cu grid with carbon supporting film and stained with 2% phosphotungstic acid. Excess staining reagent was removed using filter paper, and the grid was dried prior to transmission electron microscopy measurements (Hitachi H-7650, Japan) at 100 kV.

Cell viability. Neural cells were maintained in MEM (Gibco 11095-080) supplemented with 10% FBS and 1×MEM NEAA (Gibco 11140-050). Neural SH-SY5Y cells were plated at a density of 8000 cells per well in 96-well plates (Costar 3599) and allowed to attach overnight at 37° C. in 5% CO₂ humidified air. Aβ42 (100 μM), LPS (1000 nM), SAuM (1000 nM), or colistin (1000 nM) were then added to each group medium containing 2.5% dimethyl sulfoxide (DMSO) and sterilized by using UV light. The medium was then replaced every day and the remaining mixture was incubated for 72 hours at 37° C. in 5% CO₂ humidified air. Cell viability was measured using the CCK8 (Sigma-Aldrich 96992) assay according to the manufacturer's protocol. A 0.22-μm filter (non-pyogenic, Millex®-GV) was used to remove unbound LPS, if needed, in the LPS-Aβ42 solution.

Western Blots. To assess the level of Aβ42 in the cell culture medium, Aβ42 peptides were collected from the entire volume of the cell culture medium and total cell lysates and mixed with protein sample buffer (final 0.1 M Tris-HCl, pH6.8, 10% glycerol, 2% SDS, 1% 0-mercaptoethanol, and 0.01% bromophenol blue) for Western blot analysis. The samples of cell mixture were analyzed by SDS-PAGE gel electrophoresis and transferred to a PVDF membrane (Millipore). After blocking with blocking buffer containing 5% w/v nonfat dry milk in PBST (1× phosphate-buffered saline and 0.1% Tween 20) at room temperature for 1 h, the membrane was incubated with a primary antibody diluted in the blocking buffer at 4° C. overnight. After hybridization with primary antibody, the membrane was washed three times with PBST before the addition of an HRP-conjugated secondary antibody against the primary antibody. The membrane was then washed three times with PBST before immunoreactive bands were detected by chemiluminescence (PerkinElmer) or Ponceau S staining (Bersting Technology). Primary antibodies used in this study included the following: anti-Aβ (#8243, Cell Signaling Technology), anti-LC3 (#4108, Cell Signaling Technology), anti-cathepsin B (sc-13985, Santa Cruz Biotech), anti-cathepsin D (sc-6486, Santa Cruz Biotech), and anti-GAPDH (GTX100118, GeneTex). Culture medium containing 2.5% dimethyl sulfoxide (DMSO) was used for each group.

Statistical analyses. For biologic assays, we used GraphPad Prism (v7.02) to perform one-way ANOVAs. All data were expressed as mean±SEM, and P-values of less than 0.05 were considered to be statistically significant and showed asterisks (**, P<0.01; ***, P<0.001).

The measurement of critical aggregation concentration (CAC). The CAC of LPS was measured by small-angle X-ray scattering (SAXS). The SAXS data for the sample solutions, which were collected using a Pilatus 1M-F detector, were used to extract the zero-angle intensity Iₒ (q=0) and radius of gyration R_{g} of the LPS micelles on the basis of Guinier approximation. The value of CAC was then extracted from the intercept of the linear regression fitting of the concentration-dependent zero-angle intensity Iₒ (q=0), as shown in FIGS. 7a and 7b.

### Example 1 Intermolecular Interaction in a Non-Equilibrium State

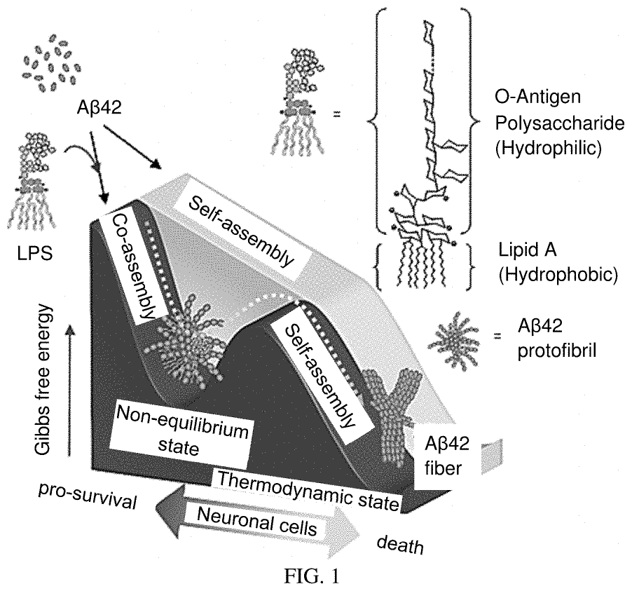

We proposed that these two supramolecules might form an intermolecular interaction in a non-equilibrium state that could subsequently impact the survival of neural cells (FIG. 1).

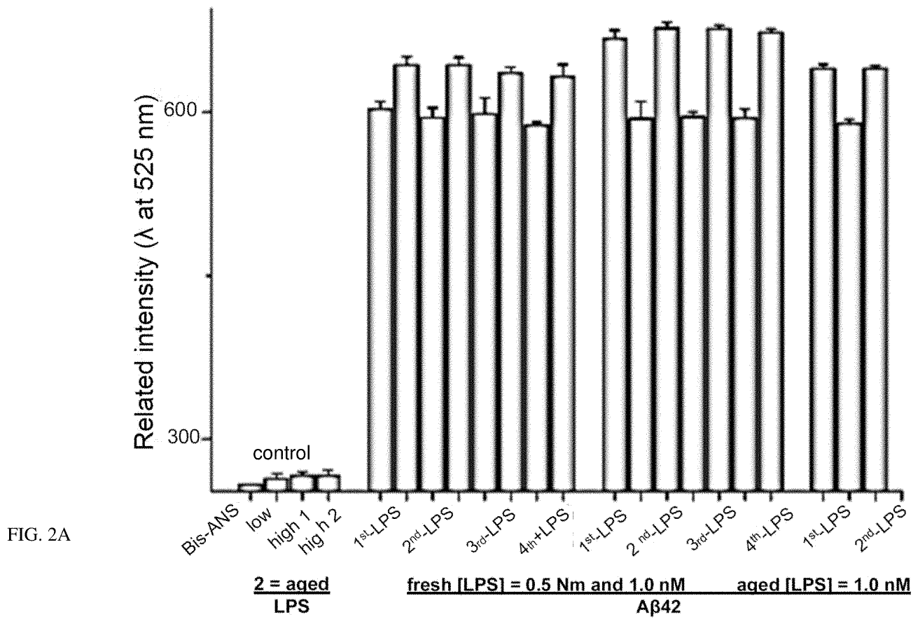

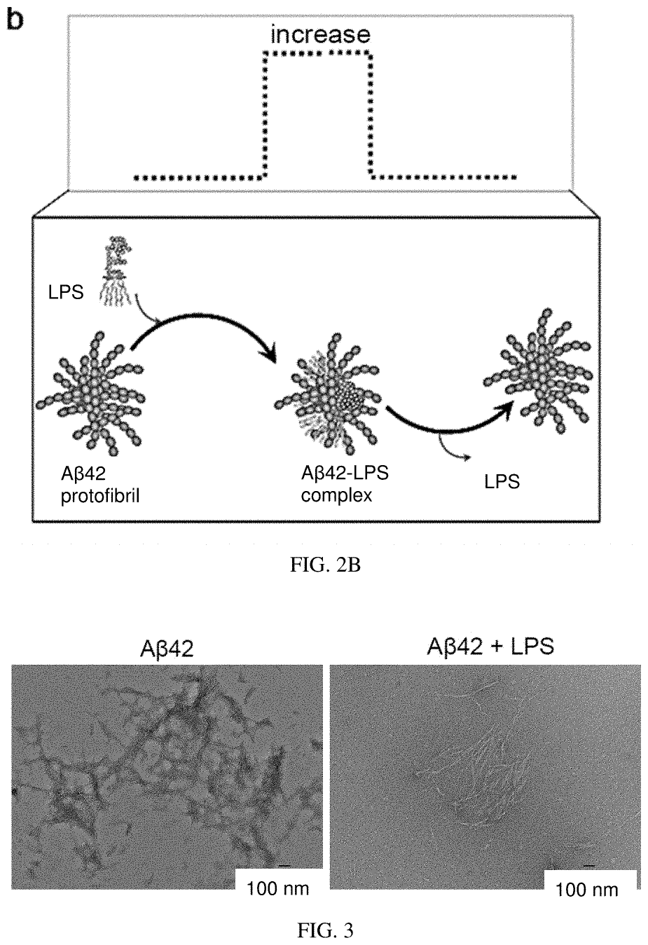

To test this hypothesis, we investigated whether LPS could form a transient complex with Aβ42 through a non-equilibrium co-assembly process that subsequently leads to dissociation. To do so, bis-ANS, a specific fluorescent dye sensitive to Aβ42 hydrophobicity (N. D. Younan, J. H. Viles, Biochemistry 2015, 54, 4297-4306), was used to assess the degree of Aβ42 hydrophobicity during the process of amyloid polymerization upon the administration of LPS. The results revealed a repeated oscillating pattern of association and dissociation between the two molecules (FIG. 2a). Specifically, the Aβ42 hydrophobicity was noticeably increased and then rapidly reduced back to a baseline level within a 30-min incubation period after adding fresh LPS. This result suggested that the Aβ42 protofibrils might induce transient LPS-Aβ42 binding when they first encounter LPS in solution (FIG. 2b). After the rapid growth of hydrophobicity, the continuing oscillation became well dampened if freshly prepared LPS was re-administered, with more pronounced effects being noted if LPS of higher concentration was re-administered. Similar effects were noted if an aged LPS (LPS being self-incubated overnight) was re-administered, but the dampening of Aβ42 hydrophobicity lessened. Since LPS is also an amphiphilic supramolecule that favors self-aggregation (K. Brandenburg, H. Mayer, M. H. J. Koch, J. Weckesser, E. T. Rietschel, U. Seydel, Eur. J. Biochem. 1993, 218, 555-563), we reasoned that aged LPS might become less competent than fresh LPS in forming LPS-Aβ42 complex. Despite the amyloid fibrils being found to continuously form in the presence of LPS over the prolonged incubation time, the formation of long fibers of Aβ42 was clearly weakened (FIG. 3). These results suggest that a dissipative non-equilibrium state of self-aggregation for Aβ42 might be induced by LPS.

### Example 2 Association Between LPS and Aβ42 Protofibrils Impacts Cytotoxicity

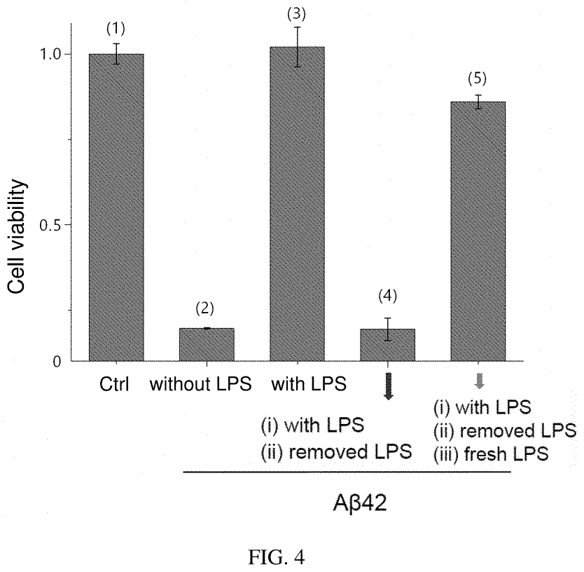

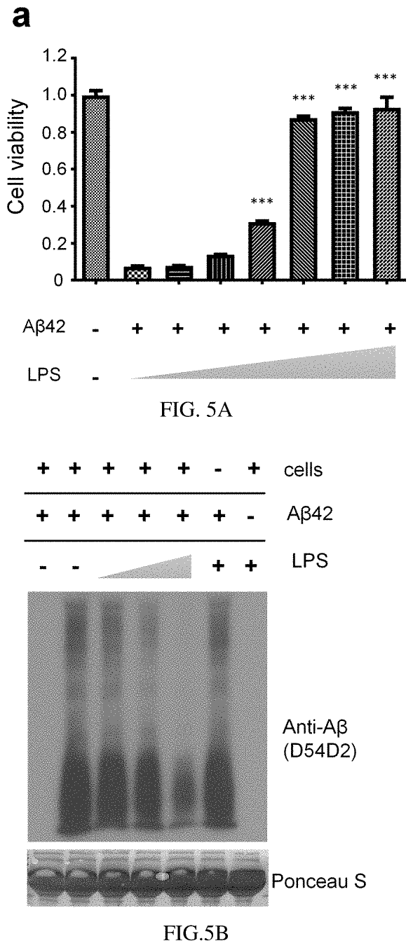

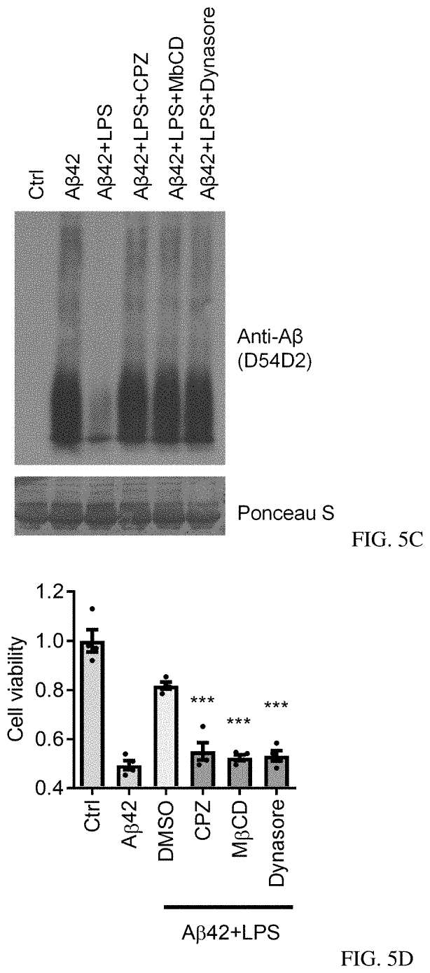

Next, it was of interest to investigate whether the transient association between LPS and Aβ42 protofibrils impacts cytotoxicity. Surprisingly, cell viability assays suggested that SH-SY5Y neuronal cells retained more than 95% cell viability after co-treatment of Aβ42 with LPS. In contrast, less than 20% cell viability was retained in cells treated with Aβ42 alone (FIG. 4, comparing columns 2 and 3). To evaluate if the rescue effect was mediated through the LPS-Aβ42 transient complex, we removed the unbound LPS from the solution and found that the rescue effect of LPS was completely lost (FIG. 4, comparing columns 3 and 4). However, when LPS were re-added back into the medium, the cell viability was returned to 85% of the normal value in control cells (FIG. 4, comparing columns 1 and 5). Moreover, the rescue effect of LPS on the Aβ42-induced apoptosis was found to occur in a dose-dependent manner with increasing LPS (FIG. 5a). That being the case, it became critical to determine this was accomplished. Under normal conditions, both the oligomeric (protofibrils) and the monomeric forms of Aβ42 can be internalized from the extracellular domains of brain cells for degradation (D. M. Walsh, B. P. Tseng, R. E. Rydel, M. B. Podlisny, D. J. Selkoe, Biochemistry 2000, 39, 10831-10839; L. A. Welikovitch, S. Do Carmo, Z. Magloczky, P. Szocsics, J. Loke, T. Freund, A. C. Cuello, Acta Neuropathol. 2018, 136, 901-917). However, during the pathogenesis of AD, the process of Aβ42 protofibril clearance via the endocytic pathway is interrupted, leading to an increased deposition of Aβ42 (C. Yu, E. Nwabuisi-Heath, K. Laxton, M. J. Ladu, Mol. Neurodegener. 2010, 5, 19; K. E. Marshall, D. M. Vadukul, K. Staras, L. C. Serpell, Cell. Mol. Life Sci. 2020). Accordingly, we speculated that the LPS-Aβ42 complex might restore the endocytic clearance of the Aβ42 peptides. To test this, we set out to investigate whether the Aβ42 levels and aggregation were both decreased. Western blot analysis of Aβ42 peptides collected from the entire volume of the cell culture medium and total cell lysates suggested that the extracellular and intracellular proteins of Aβ42 were markedly depleted upon co-treatment with LPS (FIG. 5b, lanes 3-5). However, in the absence of cells, the Aβ42 levels and oligomeric states were remained unchanged by the LPS (FIG. 5b, lanes 2 and 6), suggesting that the formation of transient complex between LPS and Aβ42 triggered a potent cellular response that resulted in the degradation of the Aβ42 peptides. These data provide strong evidence to support a role for the non-equilibrium complex of Aβ42-LPS in rescuing cells from death through promoting the clearance of Aβ42 protofibrils.

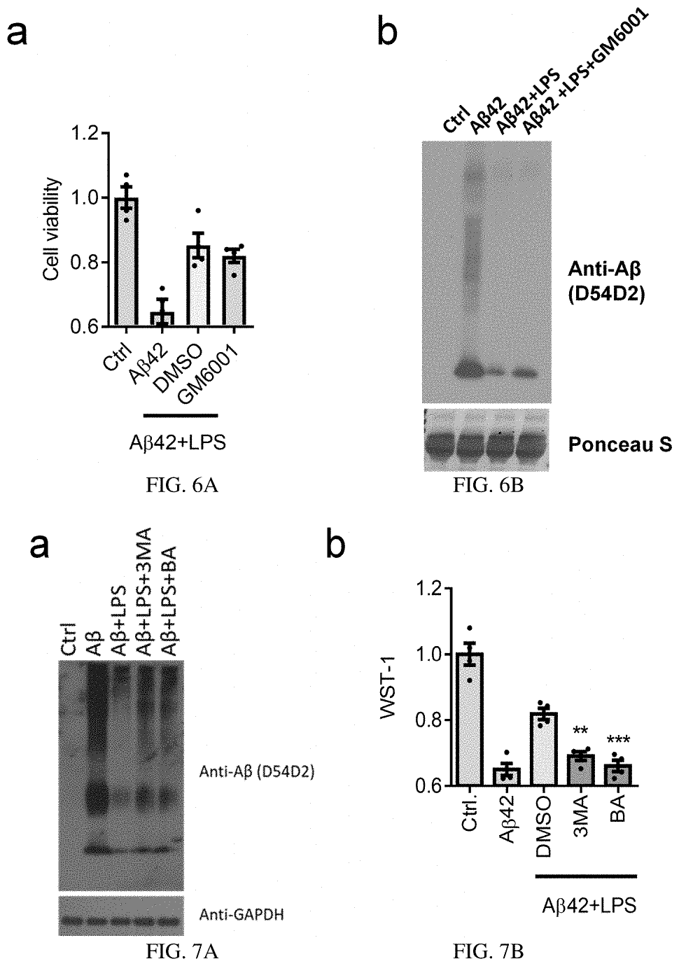

To understand the underlying mechanisms that direct this cellular process, we first tested whether matrix metalloproteinases (MMPs) secreted from cultured cells might mediate the destruction of Aβ42 peptides in the extracellular milieu. However, the treatment of neuronal cells with pan-MMP inhibitors showed no changes in Aβ42 levels in cells co-treated with LPS (FIGS. 6a and 6b). Accordingly, we turned our attention to the intracellular protein degradation pathways. We used pharmacological blockers to inhibit endocytosis mediated by the clathrin- and dynamin-dependent mechanisms. The results suggested that the blockade of the endocytic uptake of extracellular Aβ42 effectively abolished the amyloid degradation (FIG. 5c, lanes 4 to 6) as well as the pro-survival effect of LPS rescuing SH-SY5Y cells from the Aβ42-induced apoptosis (FIG. 5d). Since autophagy is also a known cellular mechanism for endo-lysosomal degradation (N. Mizushima, T. Yoshimori, B. Levine, Cell 2010, 140, 313-326), we inhibited autophagy with two typical inhibitors, methyladenine (3MA) and bafilomycin A1 (BA), and found very comparable effects to those observed for the endocytosis blockers (FIGS. 7a and 7b). The results strongly suggest that an endo-lysosomal pathway mediates the degradation of Aβ42 peptides in neuronal cells co-treated with LPS.

### Example 3 Mutual Interaction Between LPS and Soluble Aβ42 Protofibrils Occurs to Suppress the Self-Assembly Process of LPS

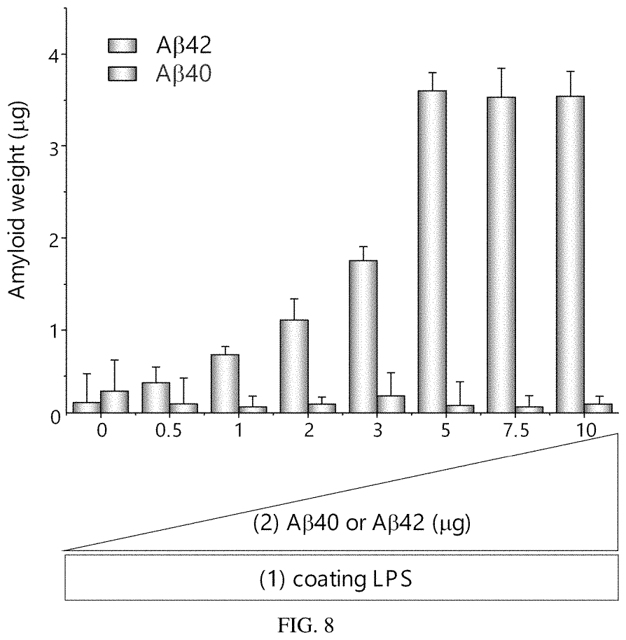

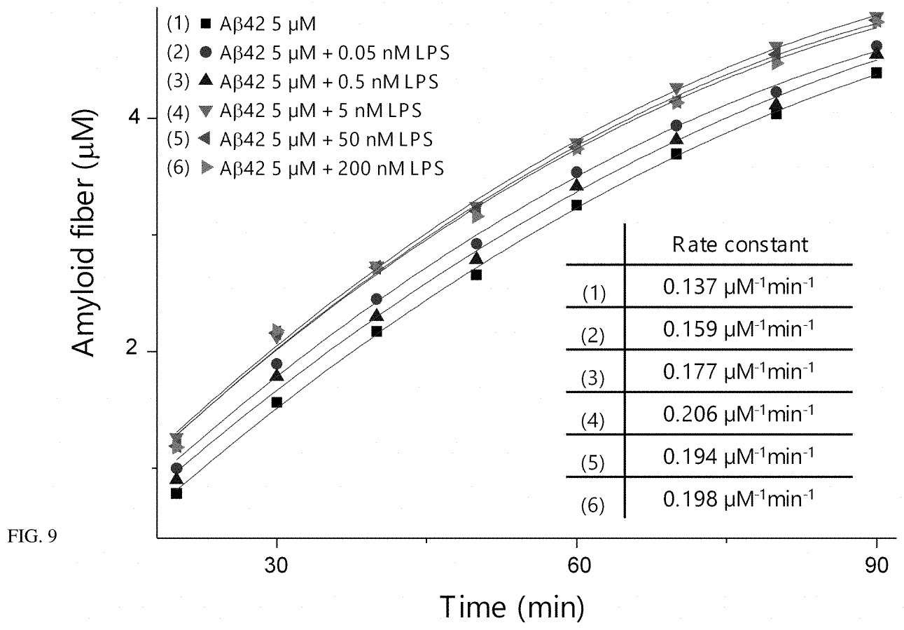

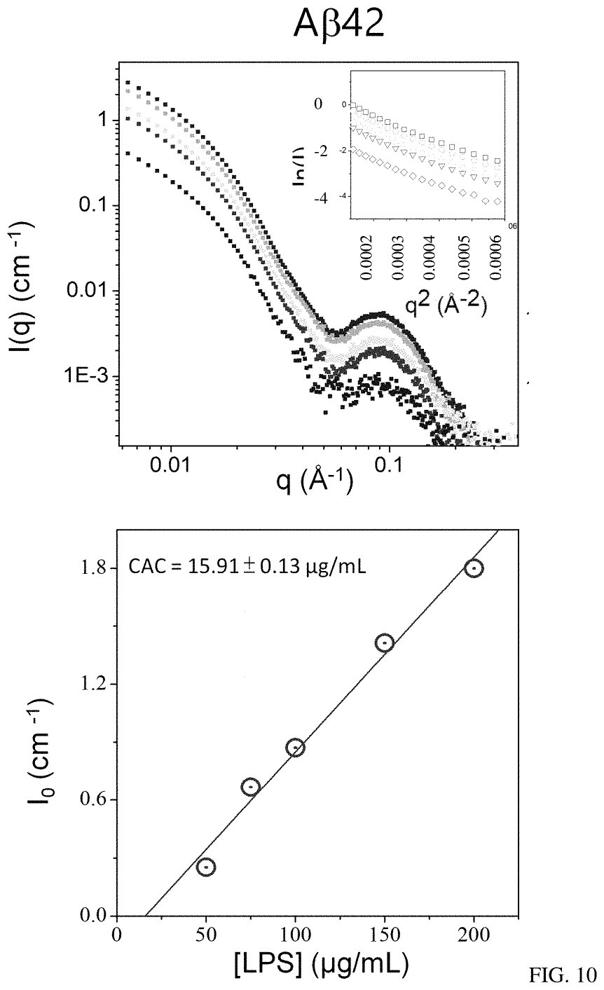

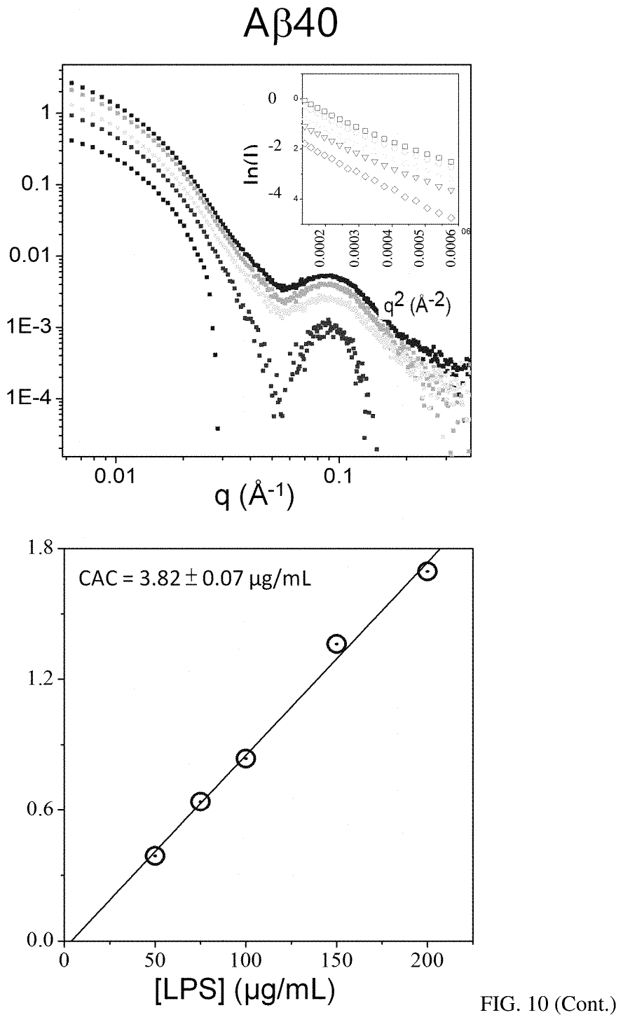

Furthermore, it was essential to clarify (i) why LPS can only bind with Aβ42 but not with Aβ40 (FIG. 8) and (ii) how they bind together. To understand the binding preference, we speculated that Aβ42 might be more prone to fast fibrillization that possibly constructs surfactant properties to increases the LPS-Aβ42 interaction in comparison to the interaction with Aβ40. As expected, Aβ42 was found to still undergo rapid fibrillization in both the presence and absence of LPS (FIG. 9), a phenomenon that was not observed for Aβ40. Additionally, small-angle X-ray scattering analysis showed that Aβ42 increased the critical aggregation concentration (CAC) of LPS from 3.49±0.052 μg/mL (F. H. Liao, T. H. Wu, Y. T. Huang, W. J. Lin, C. J. Su, U. S. Jeng, S. C. Kuo, S. Y. Lin, Nano Lett. 2018, 18, 2864-2869) to 15.91±0.13 μg/mL (FIG. 10), whereas the CAC of LPS was found to not be obviously affected by the presence of Aβ40 (3.82±0.07 μg/mL, FIG. 10). The increased CAC of LPS by Aβ42 suggested that a mutual interaction between LPS and soluble Aβ42 protofibrils occurs to suppress the self-assembly process of LPS.

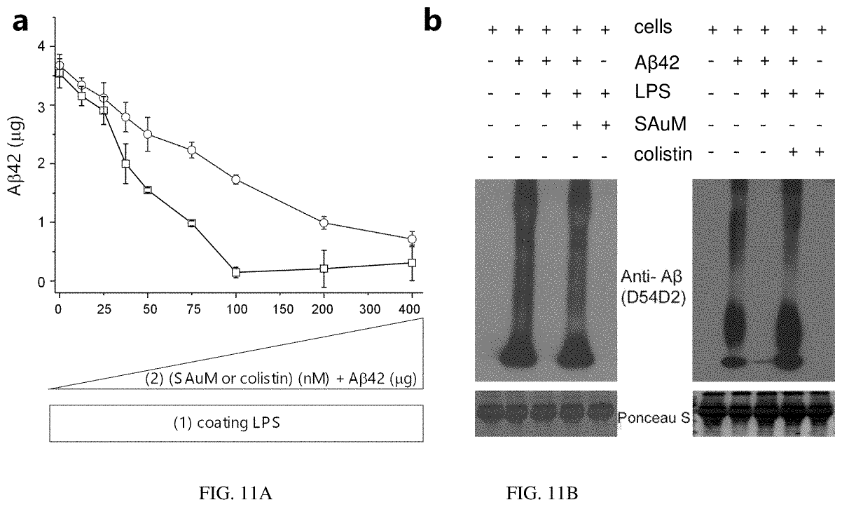

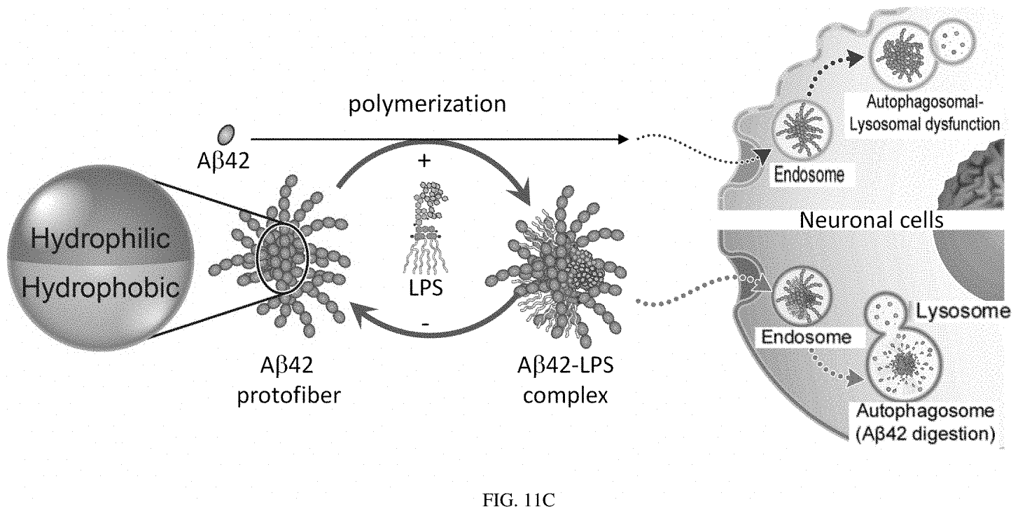

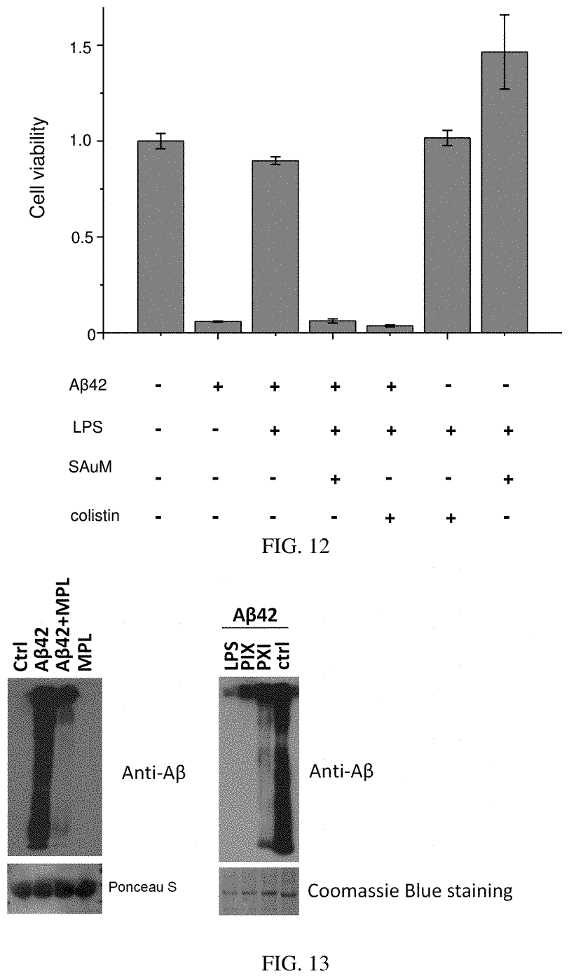

Prompted by the finding that Aβ42 formed a complex with amphiphilic LPS, we speculated that the soluble Aβ42 protofibrils might possess a specific groove that acts as a kinetic trap and enables the docking of amphiphilic LPS. To test this possibility, we used a unique LPS sequester, an atomic sheet-like gold nanocluster (identified as SAuM) with a specific dock for the lipid A of the hydrophobic domain, that has been demonstrated in our previous work (F. H. Liao, T. H. Wu, C. N. Yao, S. C. Kuo, C. J. Su, U. S. Jeng, S. Y. Lin, Angew. Chem. Int. Ed. 2020, 59, 1430-1434; P. Pristovsek, J. Kidric, J. Med. Chem. 1999, 42, 4604-4613). Indeed, the binding efficiency of Aβ42 protofibrils and LPS was found to be significantly decreased in the presence of the SAuM (FIG. 11a). Furthermore, we also showed that colistin, a cyclic peptide which is known to cap the hydrophilic domain (0-antigen) of LPS (P. Pristovsek, J. Kidric, J. Med. Chem. 1999, 42, 4604-4613), exerted a similar effect in decreasing the binding efficiency of Aβ42 protofibrils to LPS (FIG. 11a). Both results indicated that the Aβ42 protofibrils possess LPS-specific amphiphilic grooves that allow docking with the hydrophobic and hydrophilic domains of LPS. Accordingly, we abolished the complex formation of LPS and Aβ42 by blocking the lipid A or O-antigen binding sites in LPS with the two inhibitors noted. The results clearly showed that the roles of LPS in promoting the clearance of Aβ42 protofibrils (FIG. 11b) or in attenuating the cytotoxicity of Aβ42 were both compromised (FIG. 12).

We then integrated these results into a model of the structural-functional interaction between LPS and Aβ42 in modulating the endocytic clearance of Aβ42 in neuronal cells (FIG. 11c).

### Example 4 MPL, PIX and PXI can Induce Aβ42 Degradation

Although LPS reveals an efficient degradation of Aβ42 in SH-SY5Y neural cells, it still has a biosafety concern from its intricately cytotoxic immunity. We then found “Monophosphoryl lipid A (MPL, CAS 1246298-63-4, Sigma-Aldrich)” is a detoxified endotoxin lipid A fraction that belongs to one of LPS analogues, which lacks a saccharide and a phosphate group. Compared to LPS, MPL that has a controllable proinflammatory response was currently approved for clinical use in vaccine adjuvant.

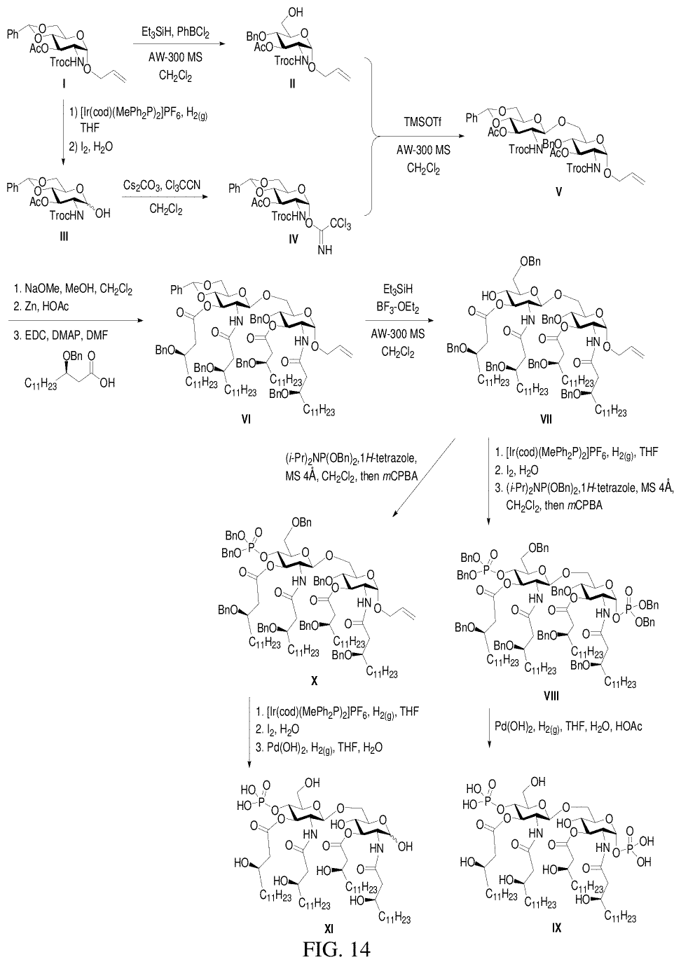

We further found “diphosphoryl or monophosphoryl lipid Aderivatives with four lipid chains (structures of IX and XI listed in FIG. 14, denoted as PIX and PXI)” is a detoxified endotoxin lipid A fraction that belongs to one of MPL analogues, which lacks two lipid chains. Compared to MPL, PIX and PXI have not cause immunity and therefore no proinflammatory response concern.

Synthesis of PIX and PXI

The synthetic routes for lipid A derivatives IX and XI are shown in FIG. 14.

Specifically, the regioselective reductive ring opening reaction of 4,6-O-benzylidene acetal of a laboratory prepared compound (I) with triethylsilane (Et₃SiH) and dichlorophenylborane (PhBCl₂) gives a desired glucosamine 6-OH acceptor (II). The imidate donor (IV) can be prepared from Compound I through a stepwise deallylation followed by trichloroacetimidate formation. Trimethylsilyl triflate (TMSOTf)-promoted glycosylation of acceptor (II) with donor (IV) provides β-(1→6)-linked disaccharide (V), which is sequentially converted into Compound VI with four lipid chains in 3 steps: (1) removal of the O-acyl groups with NaOMe liberates two hydroxy groups, (2) cleavage of the N-2,2,2-trichloroethoxycarbonyl (Troc) groups with Zn/HOAc furnishes two free amino groups, and (3) acylation of the generated hydroxy and amino groups with prepared (R)-3-benzyloxytetradecanoic acid. Compound VI is subjected to regioselective ring opening with Et₃SiH/Boron trifluoride diethyl etherate (BF₃.Et₂O) to afford the 4′-OH disaccharide VII. Subsequently, the allyl group at the anomeric position is removed, and the simultaneous phosphitylation is achieved with phosphoramidite in the presence of 1H-tetrazole, followed by oxidation with meta-chloroperoxybenzoic acid (mCPBA) to provide 1,4′-O-diphosphorylated compound VIII. Finally, deprotection of the benzyl ether is accomplished by hydrogenolysis with Pd(OH)₂ under H_{2(g)}, delivering the target molecule IX. On the other hand, the lipid A derivative XI can be synthesized from intermediate VII through phosphorylation, deallylation, and hydrogenolysis.

While the present disclosure has been described in conjunction with the specific embodiments set forth above, many alternatives thereto and modifications and variations thereof will be apparent to those of ordinary skill in the art. All such alternatives, modifications and variations are regarded as falling within the scope of the present disclosure.

What is claimed is:

1. A method for treating or preventing or delaying the onset or progression of an amyloidogenic disease in a subject in need, comprising administering a therapeutically effective amount of amphiphilic liposaccharide as an active ingredient or a pharmaceutical composition comprising the same to the subject.

2. The method of claim 1, wherein the amphiphilic liposaccharide comprises a lipid A and oligosaccharide.

3. The method of claim 1, wherein the amphiphilic liposaccharide is lipopolysaccharide (LPS), monophosphoryl lipid A (MPL), 2-deoxy-6-O-(2-deoxy-3-O-[(3R)-3-hydroxytetradecanoyl]-2-{[(3R)-3-hydroxytetradecanoyl]amino}-4-O-phosphono-β-D-glucopyranosyl)-3-O-[(3R)-3-hydroxytetradecanoyl]-2-{[(3R)-3-hydroxytetradecanoyl]amino}-1-O-phosphono-α-D-glucopyranose (PIX), or 2-deoxy-6-O-(2-deoxy-3-O-[(3R)-3-hydroxytetradecanoyl]-2-{[(3R)-3-hydroxytetradecanoyl]amino}-4-O-phosphono-β-D-glucopyranosyl)-3-O-[(3R)-3-hydroxytetradecanoyl]-2-{[(3R)-3-hydroxytetradecanoyl]amino}-D-glucopyranose (PXI) or salts thereof.

4. The method of claim 1, wherein the method is for the clearance of amyloid-beta 42 (Aβ42).

5. The method of claim 1, wherein the method is for triggering non-equilibrium co-assembly of amyloid-beta 42.

6. The method of claim 1, wherein the method is for retaining neuronal cell viability.

7. The method of claim 1, wherein the method is for rescuing Aβ42-induced apoptosis.

8. The method of claim 1, wherein the method is for enhancing endo-lysosomal clearance of amyloid-beta 42.

9. The method of claim 1, wherein the amyloidogenic disease is selected from the group consisting of Alzheimer's disease (AD), mild cognitive impairment, Parkinson's disease with dementia, Down's syndrome, diffuse Lewy body (DLB) disease, cerebral amyloid angiopathy (CAA), vascular dementia, and mixed dementia.

10. The method of claim 1, wherein the treatment or prevention or delay of the onset or progression of an amyloidogenic disease is through a clearance of amyloid-beta 42 in the subject.

11. The method of claim 1, wherein the treatment or prevention or delay of the onset or progression of an amyloidogenic disease is via triggering non-equilibrium co-assembly of amyloid-beta 42 in the subject.

12. A method for selecting an agent for treating or preventing or delaying the onset or progression of an amyloidogenic disease, comprising contacting the agent with a neuronal cell, wherein if the agent enhances clearance of amyloid-beta 42 or triggers non-equilibrium co-assembly of amyloid-beta 42, the agent is a candidate agent for treating or preventing or delaying the onset or progression of an amyloidogenic disease.

13. The method of claim 12, wherein the agent retains neuronal cell viability.

14. The method of claim 12, wherein the agent rescues Aβ42-induced apoptosis.

15. The method of claim 12, wherein the agent enhances endo-lysosomal clearance of amyloid-beta 42.

16. The method of claim 12, wherein the agent is an amphiphilic liposaccharide.

17. The method of claim 12, wherein the agent comprises a lipid A and an oligosaccharide.

18. The method of claim 12, wherein the agent is a lipopolysaccharide, monophosphoryl lipid A, 2-deoxy-6-O-(2-deoxy-3-O-[(3R)-3-hydroxytetradecanoyl]-2-{[(3R)-3-hydroxytetradecanoyl]amino}-4-O-phosphono-β-D-glucopyranosyl)-3-O-[(3R)-3-hydroxytetradecanoyl]-2-{[(3R)-3-hydroxytetradecanoyl]amino}-1-O-phosphono-α-D-glucopyranose, or 2-deoxy-6-O-(2-deoxy-3-O-[(3R)-3-hydroxytetradecanoyl]-2-{[(3R)-3-hydroxytetradecanoyl]amino}-4-O-phosphono-β-D-glucopyranosyl)-3-O-[(3R)-3-hydroxytetradecanoyl]-2-{[(3R)-3-hydroxytetradecanoyl]amino}-D-glucopyranose, or salts thereof.

19. The method of claim 12, wherein the amyloidogenic disease is selected from the group consisting of Alzheimer's disease, mild cognitive impairment, Parkinson's disease with dementia, Down's syndrome, diffuse Lewy body disease, cerebral amyloid angiopathy, vascular dementia, and mixed dementia.

20. A liposaccharide of 2-deoxy-6-O-(2-deoxy-3-O-[(3R)-3-hydroxytetradecanoyl]-2-{[(3R)-3-hydroxytetradecanoyl]amino}-4-O-phosphono-β-D-glucopyranosyl)-3-O-[(3R)-3-hydroxytetradecanoyl]-2-{[(3R)-3-hydroxytetradecanoyl]amino}-1-O-phosphono-α-D-glucopyranose, or 2-deoxy-6-O-(2-deoxy-3-O-[(3R)-3-hydroxytetradecanoyl]-2-{[(3R)-3-hydroxytetradecanoyl]amino}-4-O-phosphono-β-D-glucopyranosyl)-3-O-[(3R)-3-hydroxytetradecanoyl]-2-{[(3R)-3-hydroxytetradecanoyl]amino}-D-glucopyranose, or salts thereof.
